(19) United States
(12) Patent Application Publication
(10) Pub. No.: US 2015/0114391 A1
(43) Pub. Date: Apr. 30, 2015

(54) DEVICE FOR DISPENSING A FLUID PRODUCT

(71) Applicant: APTAR FRANCE SAS, Le Neubourg (FR)

(72) Inventors: Arnaud Colomb, Verneuil Sur Seine (FR); Maxime Kirniak, Rouen (FR)

(73) Assignee: APTAR FRANCE SAS, Le Neubourg (FR)

(21) Appl. No.: 14/397,879

(22) PCT Filed: May 30, 2013
(86) PCT No.: PCT/FR2013/051216

§ 371 Date: Oct. 30, 2014

(30) Foreign Application Priority Data
May 31, 2012 (FR) 1255010

(51) Int. Cl. A61M 15/00

(52) U.S. Cl. CPC A61M 15/0058; A61M 15/0021; A61M 15/0051; A61M 15/0075; A61M 15/0086; A61M 15/0091; A61M 2202/064; A61M 2205/19
USPC 128/203.15

(57) ABSTRACT

A fluid dispenser device including at least one individual reservoir containing a single dose of fluid, such as powder, opening means (80) being provided for opening an individual reservoir each time the device is actuated, said device including movable support means (50) that are adapted to move an individual reservoir against said opening means (80) on each actuation, said movable support means (50) being displaceable between a non-dispensing position and a dispensing position, said movable support means (50) being urged towards their dispensing position by resilient means (70), such as a spring, and being held in their non-dispensing position by blocking means that are released by the user inhaling, said device including a cocking member (800) that co-operates with said resilient means (70) and with a cam surface (51) that is formed on said movable support means (50), said cam surface (51) comprising at least one loader portion (511; 511′) for loading said resilient means (70), at least one end portion (515; 515′) for blocking said cocking member (800) in said open position and/or in said closed position of a cover element (11, 12), and at least one safety portion (513; 513′) for interconnecting at least one loader portion (511; 511′) with at least one end portion (515; 515′), said cocking member being displaceable over said safety portion (513; 513′) without additional compression or decompression of said resilient means (70).

The present invention relates to a fluid dispenser device, and more particularly to a dry-powder inhaler.

Inhalers are well known in the prior art. Various types exist. A first type of inhaler contains a reservoir receiving many doses of powder, the inhaler being provided with metering means making it possible, on each actuation, to remove one dose of said powder from the reservoir, so as to bring said dose into an expulsion duct in order to be dispensed to the user. Inhalers including individual reservoirs, such as capsules, that are loaded into the inhaler just before said reservoir is used are also described in the prior art. The advantage of such devices is that it is not necessary to store all of the doses inside the appliance, such that said appliance can be compact. However, the inhaler is more difficult to use, since the user is obliged to load a capsule into the inhaler before each use. Another type of inhaler consists in placing the doses of powder in individual predosed reservoirs, then in opening one of the reservoirs each time the inhaler is actuated. That implementation seals the powder more effectively since each dose is opened only when it is about to be expelled. In order to make such individual reservoirs, various techniques have already been proposed, such as an elongate blister strip or blisters disposed on a rotary circular disk. All existing types of inhalers, including those described above, present both advantages and drawbacks associated with their structures and with their types of operation. Thus, with certain inhalers, there is the problem of metering accuracy and reproducibility on each actuation. In addition, the effectiveness of the dispensing, i.e. the fraction of the dose that effectively penetrates into the user's lungs in order to have a beneficial therapeutic effect, is also a problem that exists with a certain number of inhalers. A solution for solving that specific problem has been to synchronize the expulsion of the dose with the inhalation of the patient. Once again, that can create drawbacks, in particular in that type of device, the dose is generally initially loaded into an expulsion duct before inhalation, then expulsion is synchronized with inhalation. That means that if the user drops, shakes, or manipulates the inhaler in an undesirable or inappropriate manner between the moment when the user loads the dose (either from a multidose reservoir or from an individual reservoir) and the moment when the user inhales, then the user risks losing all or part of the dose, with said dose possibly being spread about inside the appliance. In that event, there can exist a high risk of overdosing the next time the device is used. The user who realizes that the dose is not complete will load a new dose into the appliance, and while the new dose is being inhaled, a fraction of the preceding dose that was lost in the appliance could thus be expelled at the same time as the new dose, thereby causing an overdose. In the treatments envisaged, such overdosing can be very harmful, and the authorities in all countries are issuing ever-stricter requirements to limit the risk of overdosing as much as possible. Another problem that may occur relates to assembling certain parts, in particular movable parts, that need to withstand large stresses in operation, and for which assembly needs to be particularly reliable so as to avoid any risk of malfunctioning. With the small size of certain parts, it can be complicated to guarantee such reliable assembly. With inhalers that are primed, e.g. while opening, then triggered by inhalation, it is important to avoid or to limit the risks of accidental triggering after priming and before inhalation, without imposing a triggering inhalation threshold that is too high, which could be difficult for weak people to achieve.

An object of the present invention is to provide a fluid dispenser device, in particular a dry-powder inhaler, that does not have the above-mentioned drawbacks.

In particular, an object of the present invention is to provide such a device that is simple and inexpensive to manufacture and to assemble, that can be assembled and used reliably, guaranteeing metering accuracy and reproducibility on each actuation, providing an optimum yield with regard to the effectiveness of the treatment, by making it possible to dispense a substantial fraction of the dose to the zones to be treated, in particular the lungs, avoiding, in safe and effective manner, any risk of overdosing, and that is as compact as possible, while guaranteeing sealing and absolute integrity of all of the doses up to their expulsion.

The present invention thus provides a fluid dispenser device including a main body and at least one cover element that is mounted to pivot on said main body between a closed position and an open position, said device including at least one individual reservoir containing a single dose of fluid, such as powder, opening means being provided for opening an individual reservoir each time the device is actuated, said device including movable support means that are adapted to move an individual reservoir against said opening means on each actuation, said movable support means being displaceable between a non-dispensing position and a dispensing position, said movable support means being urged towards their dispensing position by resilient means, such as a spring, and being held in their non-dispensing position by blocking means that are released by the user inhaling, said device including a cocking member that co-operates with said resilient means and with a cam surface that is formed on said movable support means, said cam surface comprising at least three cam portions with slopes that are different, having at least one loader portion for loading said resilient means, at least one end portion for blocking said cocking member in said open position and/or in said closed position of said at least one cover element, and at least one safety portion for interconnecting at least one loader portion with at least one end portion, said cocking member being displaceable over said safety portion without additional compression or decompression of said resilient means.

Advantageously, said at least one loader portion is of shape that is plane and rectilinear.

Advantageously, said at least one safety portion is in the shape of a circular arc, such that no sideways force is exerted on said cocking member while it is co-operating with said safety portion.

Advantageously, said at least one end portion is plane or V-shaped.

Advantageously, starting from the closed position, said cam surface comprises a loader portion, a safety portion, and an end portion.

Advantageously, the device includes an inhalation trigger system that comprises a deformable air chamber that co-operates with an inhalation piece, and a trigger element that co-operates with said air chamber, such that during inhalation through said inhalation piece, said air chamber is deformed and said trigger element actuates said opening means, such that during inhalation through the inhalation piece, a reservoir is opened by said opening means.

Advantageously, said opening means include a perforator element that is stationary relative to said main body and that is adapted to cut a closure wall of the reservoir in such a manner that the cut portion(s) does/do not obstruct the opening(s) that is/are formed.

These characteristics and advantages and others of the present invention appear more clearly from the following detailed description, given by way of non-limiting example, and with reference to the accompanying drawing, and in which:

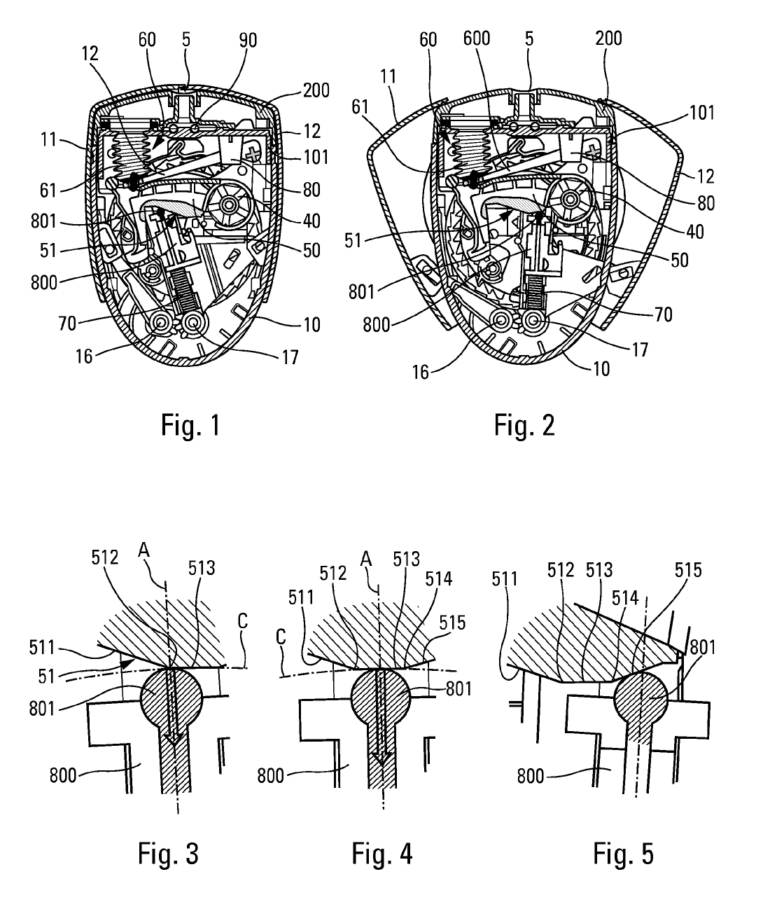

FIGS. 1 and 2 are diagrammatic section views of a dispenser device in a first embodiment of the invention, respectively in the closed and open positions of the covers;

FIG. 3 is a diagrammatic view of a detail of the cocking member in a first intermediate position, between the closed and open positions of the covers;

FIG. 4 is a view similar to the view in FIG. 2, in a second intermediate position;

FIG. 5 is a view similar to the views in FIGS. 2 and 3, in the open position of the covers;

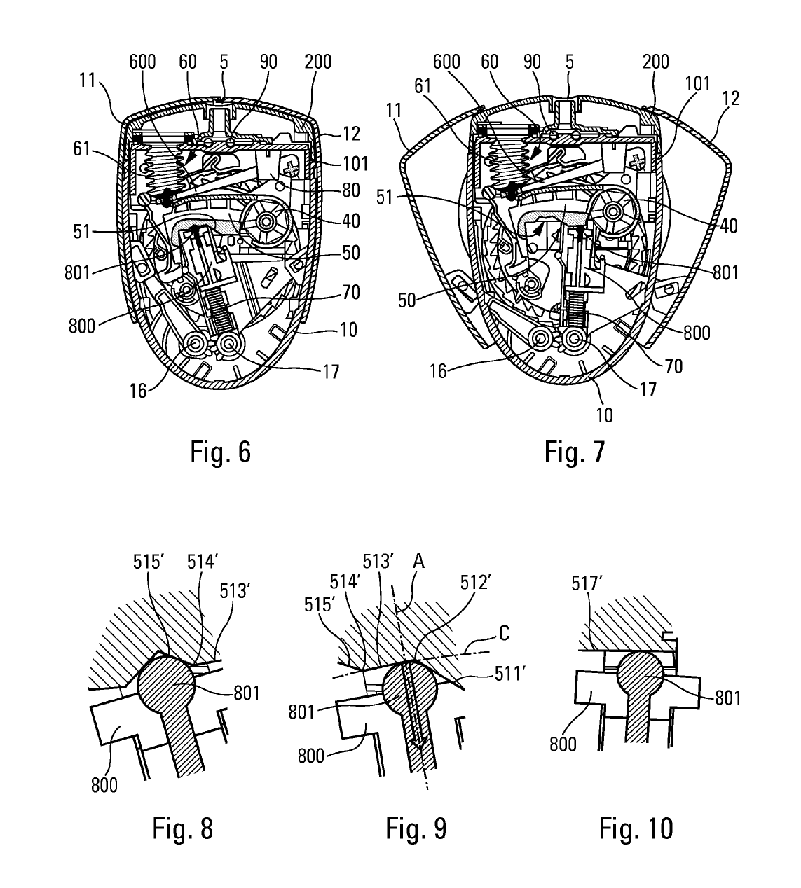

FIGS. 6 and 7 are views similar to the views in FIGS. 1 and 2, showing a second embodiment of the invention;

FIGS. 8 to 10 are views similar to the views in FIGS. 3 to 5, showing the FIGS. 6 and 7 device respectively in the closed, intermediate, and open positions of the covers; and

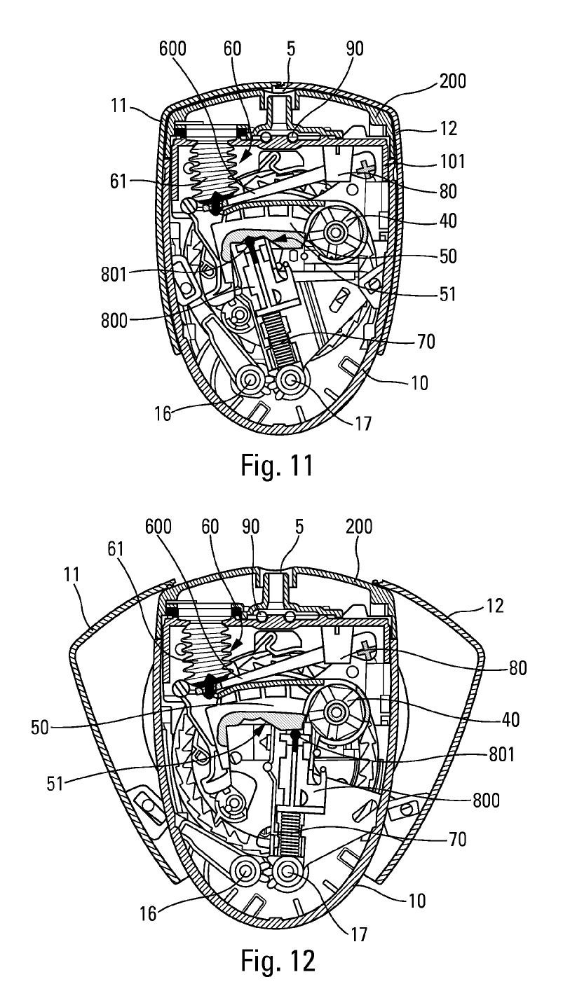

FIGS. 11 and 12 are views similar to the views in FIGS. 1 and 2, showing a third embodiment of the invention.

FIGS. 1 and 2 show an advantageous embodiment of a dry-powder inhaler. The inhaler includes a main body 10 on which there can be slidably mounted two cover-forming portions 11, 12 that are adapted to be opened so as to open and prime the device. The main body 10 can be approximately rounded in shape, as shown in the figures, but it could be of any other appropriate shape. An upper body 101 is assembled to the main body 10, and a mouthpiece 200 is assembled on said upper body 101. The mouthpiece 200 defines a dispenser orifice 5 through which the user inhales while the device is being actuated. The dispenser orifice 5 is typically arranged approximately in the center of the mouthpiece 200. The covers 11, 12 can open by pivoting about a common pivot axis, or about two parallel axes by being meshed together. Any other opening means for opening the device can be envisaged. In a variant, the device could include only a single cover instead of two.

Inside the main body 10 there is provided a strip (not shown for the sake of clarity) of individual reservoirs, also known as blisters, said strip being made in the form of an elongate strip on which the blisters are disposed one behind another, in manner known per se. The blister strip is advantageously constituted by a base layer or wall that forms the cavities receiving the doses of powder, and by a closure layer or wall that covers each of said blisters in sealed manner. Before first use, the blister strip can be rolled-up inside the main body 10, preferably in a storage portion, and first strip displacement means 40, in particular rotary means, are provided for progressively unrolling the blister strip and for causing it to advance.

Second displacement means 50, in particular means that are mounted to pivot on the main body 10, are provided for bringing a respective blister into a dispensing position each time the device is actuated. The second displacement means are advantageously mounted to pivot between a non-dispensing position and a dispensing position in which a blister co-operates with said opening means.

The strip portion including the empty blisters is advantageously adapted to be rolled-up at another location of said body 10, preferably a reception portion, as described in greater detail below.

The inhaler includes blister opening means 80 (shown only very diagrammatically for the sake of clarity) preferably comprising a perforator and/or cutter needle for perforating and/or cutting the closure layer of the blisters. Preferably, the opening means comprise a perforator element 80 that is stationary relative to the main and upper bodies 10, 101, and against which a respective blister is displaced on each actuation by the second displacement means. The blister is thus perforated by said perforator element that penetrates into said blister so as to expel the powder by means of the user inhaling. Advantageously, the perforator element is adapted to cut a closure wall of the reservoir in such a manner that the cut portion(s) does/do not obstruct the opening(s) that is/are formed. Documents WO 2006/079750 and WO 2009/007640 describe such blister opening means, and they are thus incorporated in the present description by way of reference.

The first displacement means 40 are adapted to cause the blister strip to advance after each inhalation of the user. The second displacement means 50 are adapted to displace the blister to be emptied against said opening means during actuation, before each inhalation. The second displacement means can be urged by a resilient element 70, such as a spring or any other equivalent resilient element, said resilient element being suitable for being prestressed while the device is being opened.

Preferably, the first displacement means 40 are formed by an indexer wheel that receives and guides the blister strip. The description below is thus made with reference to such an indexer wheel 40. Turning the indexer wheel 40 causes the blister strip to advance. Before each inhalation, a full blister is always in a position facing the opening means 80. The second displacement means 50 can include a pivot member that is mounted to pivot about a pivot axis, said indexer wheel 40 advantageously being rotatably mounted on said pivot member.

An actuation cycle of the device can be as follows. During opening of the device, the two cover-forming lateral portions 11, 12 are moved away from each other by pivoting about the body so as to open the device and thus spring-load the device. In this position, the indexer wheel 40 cannot be displaced towards the perforator element 80, since the second displacement means 50 are held by appropriate blocking means (not shown for the sake of clarity). Documents WO 2009/077700 and WO 2009/136098 describe such blocking means, and they are thus incorporated in the present description by way of reference. While the user is inhaling through the mouthpiece, the blocking means are unblocked, thereby causing said indexer wheel 40 to move towards the needle, and thereby causing a blister to be opened.

As explained above, it is desirable for the opening means to be actuated by the user inhaling. In order to trigger the opening means by inhalation, an inhalation trigger system 60 is provided that advantageously comprises an air chamber 61 that is deformable under the effect of inhalation, the air chamber being adapted to release the blocking means. The air chamber 61 may advantageously be made in the form of a bellows. Inhalation by the user causes said deformable air-chamber to deform, thereby releasing said blocking means and enabling the second displacement means to be displaced, and therefore enabling a respective blister to be displaced towards its opening position. The blister is therefore opened only on inhalation, such that it is emptied simultaneously. Thus, there is no risk of any of the dose being lost between opening the blister and emptying it.

The inhaler further includes a dispersion chamber 90 for receiving the dose of powder after a respective blister has been opened. The dispersion chamber is advantageously provided with at least one and preferably more beads that are displaced inside said chamber 90 during inhalation, in particular so as to improve dispensing of the air and powder mixture after a blister has been opened, in order to increase the effectiveness of the device.

It can be advantageous for the opening means, in particular for the perforator element, to be connected directly to said dispersion chamber, e.g. via a channel leading to said chamber 90.

After inhalation, when the user closes the device, all of the components return to their initial, rest positions. The device is thus ready for a new utilization cycle.

In an advantageous aspect of the inhaler, the blisters are formed on a flexible elongate strip that, initially, is mainly stored in the form of a roll in a storage housing inside the main body 10 of the device. Advantageously, the rolled-up blister strip is held by inner walls of said storage housing without its rear end (rear in the advancement direction of the blister strip) being fastened relative to said main body 10, thereby enabling the blister-strip roll to be assembled more easily inside the device. The blister strip is displaced by means of the indexer wheel 40 that advantageously presents at least one and preferably more recesses, each having a shape that corresponds to the shape of the blisters. Thus, when the indexer wheel 40 turns, it causes the blister strip to advance. Naturally, in a variant or in additional manner, it is possible to use other means for advancing the blister strip, e.g. providing a profile on the longitudinal lateral edges of the blister strip, said profile being adapted to co-operate with appropriate drive means. In addition, holes formed along the lateral edges of the blister strip could also be used to cause the blister strip to advance by means of sprocket wheels co-operating with said holes.

After opening one or more blisters, the blister-strip portion with the empty blisters must be suitable for being stored in easy and compact manner in the device, while avoiding any risk of blockage. Advantageously, the used blister strip is rolled-up automatically, once again forming a roll.

In still another aspect of the inhaler, a dose counter or indicator device (not shown for the sake of clarity) may also be provided. The device may include numbers or symbols that are marked directly on the blister strip, and that are visible through an appropriate window in the main body 10 of the device. In a variant, it is possible to envisage using a counter with one or more rotary disks or rings including numbers or symbols. Documents WO 2008/012458 and WO 2011/154659 describe such counters, and they are thus incorporated in the present description by way of reference. An object of the invention is to avoid counting doses that have not been dispensed, e.g. in the event of a manipulation error, or of an incomplete manipulation of the device. It is thus desirable that the counter or indicator is actuated only once the user has inhaled, since it is this inhalation that makes it possible for the blister to open and the dose contained therein to be dispensed. Advantageously, the counter is thus actuated after inhalation, when the user closes the device.

The movable cover element 12 is secured to a cocking member 800 that can slide in an appropriate housing. The cocking member 800 thus advantageously pivots relative to said body 10 together with the cover element 12. The cocking member 800 may be moved against a spring 70, advantageously a coil spring, that is arranged in said housing. The cocking member 800 is thus connected at one end to said spring 70, and at the other end it co-operates with the second displacement means, in particular with a pivot member 50 that is mounted to pivot on the body 10, and on which the indexer wheel 40 is fastened is rotary manner.

When the movable cover element 12 is opened, the cocking member 800 is displaced in its housing, compressing the spring 70. The pivot member 50 of the second displacement means is itself prevented from moving by the above-mentioned blocking means that are released only at the moment of inhalation. Thus, in the absence of any inhalation in the open position, closing the cover elements 11, 12 would merely cause the cocking member 800 to return to its rest position and the spring 70 to decompress.

Thus, by opening the inhaler, the user primes the system. If the user does not inhale and closes the inhaler, said inhaler merely returns to its start position without displacing the blister strip or the blocking means. There is thus no risk of a blister (and thus an active dose of substance) being lost by accidental or incomplete actuation in which the user does not inhale between opening and closing. Opening the blister, emptying it, dispensing the powder into the lungs of the user, displacing the blister strip to bring a new full blister to face the opening means, and counting the dose are thus possible only if the user inhales.

The blocking means that block the second displacement means and in particular the pivot member that co-operates with the cocking member, are connected to the deformable air chamber 61 that is sensitive to the user inhaling, by means of a trigger element 600, so that while the user is inhaling, said deformable air chamber deforms, causing the trigger element 600 to pivot and thus causing said blocking means to be released. This enables said second displacement means to be displaced towards their dispensing position under the effect of the force exerted by the compressed spring 70 on the cocking member 800 that pushes against the pivot member 50. Such displacement causes a full blister to be opened and a dose to be dispensed.

A cam surface 51 is formed on said movable support means 50, on which the cocking member 800 slides. The cocking member 800 is thus adapted to compress the spring 70 when the cover element 12 is open, and to decompress said spring 70 when said cover element 12 is closed.

Advantageously, in its portion in contact with the cam surface 51, the cocking member 800 includes a rounded portion 801 for facilitating sliding of the cocking member 800 on said cam surface 51.

In this embodiment, the movable support means are made in the form of a member 50 that is pivotally mounted on the body 10 about a pivot axis. Since the above-mentioned cam surface 51 is formed on said pivot member 50, when the spring 70 is loaded while the movable cap element 12 is opening, said pivot member 50 is urged towards its dispensing position by said cocking member 800 and the spring 70 is compressed.

After inhalation, i.e. in the dispensing position, the blocking means have been released, and the movable support means 50 have been displaced upwards by the compressed spring 70.

Advantageously, the two movable cover elements 11, 12 mesh together via appropriate gearing so as to guarantee symmetrical opening and closing of said two movable cover elements. They can mesh together in the proximity of their pivot axes 16, 17.

In the invention, the cam surface 51 comprises at least three cam portions with slopes that are different. At least one loader portion is provided so as to displace the cocking member 800 so as to load the spring 70. At least one end portion is provided so as to block the cocking member 800 in the open and/or closed position. At least one safety portion is provided, arranged directly before and/or after an end portion so as to ensure reliable operation. In the safety portion, the cocking member 800 is displaced without additional compression and/or decompression.

FIGS. 1 to 5 show a first embodiment. In this variant, the cam surface includes a loader portion 511 that is substantially rectilinear. A safety portion 513 is connected to said loader portion at a first vertex 512. The safety portion 513 is preferably in the shape of a circular arc, and is connected to an end portion 515 at a second vertex 514.

Starting from the closed position in FIG. 1, when the user opens the covers 11, 12, the cocking member 800 initially slides over the loader portion 511. This causes the cocking member 800 to slide in its housing so as to load the spring 70.

When the cocking member arrives at the first vertex 512, as shown in FIG. 3, the spring is loaded, i.e. compressed. This position is reached in a first intermediate position in which the covers 11, 12 are not completely open.

Continuing to open the covers causes the cocking member 800 to slide over the safety portion 513. The safety portion could be plane, but it is preferably in the shape of a circular arc C, with the radius of the circular arc corresponding to the radius of the pivoting movement of the cocking member 800 while the covers are opening. As a result, no sideways force is exerted on the cocking member 800 while it is displaced over the safety portion 513. Only the axial reaction to the force of the loaded spring 70 is applied to the cocking member 800 along its longitudinal axis A, as shown by the arrow in FIG. 4. If the opening or closing movement of the covers were to stop on the safety portion 513, e.g. in the second intermediate position in FIG. 4, the device would be in a stable position. In addition, as a result of the circular arc shape, the cocking member 800 no longer slides axially when it is displaced in the safety portion. The spring 70 is thus completely loaded at the end of the loader portion 511, at the first vertex 512. The end portion of opening, taking place over said safety portion 513 and up to the end portion 515, is thus easy, since no additional compression or decompression of the spring 70 is required.

When the covers are completely open, the cocking member comes to co-operate with the end portion 515. The end portion may be formed by a plane portion, as shown in FIG. 5, or it may have a V-shape, so as to block the cocking member 800 at least slightly, and thus block the covers 11, 12, in the open position of the covers.

The open position is well blocked, but nevertheless, during actuation, large forces are released which can sometimes cause the cocking member 800 to come out of the end portion 515 in untimely or unwanted manner. In such circumstances, the safety portion 513 acts to hold the device in a stable in position, enabling the inhaler to be actuated reliably. This would not occur if the end portion were connected directly to the loader portion 511.

FIGS. 6 to 10 show a second embodiment, which is substantially reversed compared to the first embodiment in FIGS. 1 to 5. In this embodiment, the end portion 515′ is at the start of the cam surface 51, in the cover-opening sequence, followed, after a vertex 514′, by the safety portion 513′, itself connected to the loader portion 511′ via another vertex 512′. The loader portion 511′ is thus connected in a final portion 517′, as can be seen in FIG. 10, that may be plane or circularly arcuate. In this embodiment, the end portion 515′ is V-shaped, as can be seen in FIG. 8, but it could also be of shape that is approximately plane.

Operation is thus similar but reversed compared to the first embodiment. It is in the open position that the cocking member 800 is blocked in the end portion 515′, which makes it possible to guarantee a good closed position for the covers.

FIGS. 11 and 12 show a third embodiment in which the first and second embodiments in FIGS. 1 to 5 and 6 to 10 are combined.

In this variant, both the open and the closed positions of the covers are well defined by end portions, and the substantially central loader portion is connected to each end portion via a respective safety portion. The advantages of the first and second embodiments are combined in the third embodiment.

The present invention therefore makes it possible to provide a dry-powder inhaler that provides the following features:
a plurality of individual doses of powder stored in individual sealed blisters, e.g. 30 or 60 doses stored on a rolled-up strip;
the powder is released by perforation that is achieved by the user inhaling, the blister being perforated by means of an inhalation detector system that is coupled to a prestressed release system;
appropriately-shaped drive means that are engaged with blisters so as to displace the blister strip after each inhalation, and bring a new full blister into a position in which it is to be opened by appropriate opening means;
means for avoiding doses being lost in the event of the inhaler being opened, but in the absence of any inhalation; and
a dose indicator adapted to count the doses only in the event of inhalation.

Other features are also provided by the device of the invention as described above.

It should be observed that the various features, even if they are shown as being provided simultaneously on the inhaler, could be implemented separately. In particular, the inhalation trigger mechanism could be used regardless of the type of reservoir opening means, regardless of the use of a dose indicator, regardless of the way in which the individual blisters are arranged relative to one another, etc. The cocking means and the inhalation trigger system could be made in some other way. The same applies for other component parts of the device.

Various modifications are also possible for the skilled person without departing from the scope of the present invention as defined in the accompanying claims. In particular, the various characteristics and functions of the device described with reference to the drawings can be combined together in any appropriate manner.

1. A fluid dispenser device including a main body and at least one cover element that is mounted to pivot on said main body between a closed position and an open position, said device including at least one individual reservoir containing a single dose of fluid, such as powder, opening means being provided for opening an individual reservoir each time the device is actuated, said device including movable support means that are adapted to move an individual reservoir against said opening means on each actuation, said movable support means being displaceable between a non-dispensing position and a dispensing position, said movable support means being urged towards their dispensing position by resilient means, such as a spring, and being held in their non-dispensing position by blocking means that are released by the user inhaling, said device including a cocking member that co-operates with said resilient means and with a cam surface that is formed on said movable support means, wherein said cam surface comprises at least three cam portions with slopes that are different, having at least one loader portion for loading said resilient means, at least one end portion for blocking said cocking member in said open position and/or in said closed position of said at least one cover element, and at least one safety portion for interconnecting at least one loader portion with at least one end portion, said cocking member being displaceable over said safety portion without additional compression or decompression of said resilient means.

2. A device according to claim 1, wherein said at least one loader portion is of shape that is plane and rectilinear.

3. A device according to claim 1, wherein said at least one safety portion is in the shape of a circular arc, such that no sideways force is exerted on said cocking member while it is co-operating with said safety portion.

4. A device according to claim 1, wherein said at least one end portion is plane or V-shaped.

5. A device according to claim 1, wherein, starting from the closed position, said cam surface comprises a loader portion, a safety portion, and an end portion.

6. A device according to claim 1, including an inhalation trigger system that comprises a deformable air chamber that co-operates with an inhalation piece, and a trigger element that co-operates with said air chamber, such that during inhalation through said inhalation piece, said air chamber is deformed and said trigger element actuates said opening means, such that during inhalation through the inhalation piece, a reservoir is opened by said opening means.

7. A device according to claim 1, wherein said opening means include a perforator element that is stationary relative to said main body and that is adapted to cut a closure wall of the reservoir in such a manner that the cut portion(s) does/do not obstruct the opening(s) that is/are formed.
